(19) United States
(12) Patent Application Publication
(10) Pub. No.: US 2024/0177838 A1
(43) Pub. Date: May 30, 2024

(54) SYSTEMS AND METHODS FOR PROCESSING ELECTRONIC IMAGES USING DEEP FOUNDATION MODELS

(71) Applicant: PAIGE.AI, Inc., New York, NY (US)

(72) Inventors: Siqi LIU, New York, NY (US); Eugene VORONTSOV, Montreal (CA); Alican BOZKURT, New York, NY (US); George SHAIKOVSKI, London (GB); Michal ZELECHOWSKI, Bolechowice (PL); Adam CASSON, New York, NY (US); Jan BERNHARD, Brooklyn, NY (US); Sid SENTHILNATHAN, New York, NY (US); Matthew LEE, London (GB); Ran GODRICH, New York, NY (US); Thomas FUCHS, New York, NY (US); Brandon ROTHROCK, Los Angeles, CA (US)

(21) Appl. No.: 18/521,903

(22) Filed: Nov. 28, 2023

Related U.S. Application Data
(60) Provisional application No. 63/385,364, filed on Nov. 29, 2022.

(51) Int. Cl. G16H 30/40; G06T 7/00; G06T 7/11; G06T 9/00; G06V 10/764; G16H 30/20; G16H 50/20; G16H 50/70

(52) U.S. Cl. CPC G16H 30/40; G06T 7/0012; G06T 7/11; G06T 9/00; G06V 10/764; G16H 30/20; G16H 50/20; G16H 50/70

(57) ABSTRACT

Systems and methods for processing digital medical images to infer metadata from those images are disclosed. In some aspects, digital medical images may be processed to infer metadata by receiving a plurality of digital medical images, receiving a prompt, the prompt being a request for a specific type of metadata to be inferred from the plurality of digital medical images, determining, using a trained foundation model, at least one feature descriptor from the plurality of digital medical images based on the prompt, and providing for output the at least one feature descriptor for each of the plurality of digital medical images.

### CROSS-REFERENCE TO RELATED APPLICATIONS

This patent application claims the benefit of U.S. Provisional Application No. 63/385,364, filed on Nov. 29, 2022, the entirety of which is incorporated by reference herein

### FIELD OF THE DISCLOSURE

Various embodiments of the present disclosure relate generally to large-scale image processing. More specifically, particular embodiments of the present disclosure relate to systems and methods for large-scale image processing using deep foundation models to infer metadata from the images.

### BACKGROUND

Generally, analysis of large quantities of data, e.g., using machine learning systems, may be limited by annotation requirements, variable tissue types within samples, etc. Furthermore, to capture the full diversity of complex domains, models need to be considerably larger in terms of parameter complexity, requiring extremely large data sets to train on. Training systems to analyze large, variable, and/or unannotated data may require vast amounts of compute to train, especially when the data includes high-resolution images, such as those used in computational pathology. Other challenges may include a lack of data, even if that data does not need exhaustive annotations. Further, even when utilizing supervised or weakly supervised training methods, the ability to generalize between applications may be limited, the availability of clinical labels or manual annotations may be reduced, and the training may generalize poorly with long tail distribution and rare events. Conventional techniques, including the foregoing, fail to account for the need to analyze large quantities of data, often across various modalities and without annotations. Systems and/or methods that operate in a pan-cancer and/or pan-tissue manner are needed.

The foregoing general description and the following detailed description are exemplary and explanatory only and are not restrictive of the disclosure. The background description provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

According to certain aspects of the disclosure, methods and systems are disclosed for generating and modifying foundation models. Each of the aspects of the disclosure herein may include one or more of the features described in connection with any of the other disclosed aspects.

According to one example of the present disclosure, methods for processing digital medical images to infer metadata from those images, may be described. An exemplary method may include receiving a plurality of digital medical images, receiving a prompt, the prompt being a request for a specific type of metadata to be inferred from the plurality of digital medical images, determining, using a trained foundation model, at least one feature descriptor from the plurality of digital medical images based on the prompt, and causing to, or providing for, output the one or more feature descriptors for each of the plurality of digital medical images.

According to another example of the present disclosure, methods for training a foundation model to process digital medical images to infer metadata from those images may be described. An exemplary method may include receiving a plurality of digital medical images, generating a plurality of image tokens from the digital medical images, the image tokens being fixed-sized patches, removing a subset of the plurality of image tokens from each of the digital medical images to generate a remaining plurality of image tokens from each of the digital medical images, encoding, using an encoder, the remaining plurality of image tokens from each of the digital medical images, adding a classification token to the encoded image tokens, appending masked tokens with position encodings to each respective encoded image token, and reconstructing, using a decoder, the image tokens, such that the image tokens align with original image pixel values.

According to another example of the present disclosure, systems for processing digital medical images to infer metadata from those images. An exemplary system may include at least one memory storing instructions and at least one processor configured to execute the instructions to perform operations. The operations may include receiving a plurality of digital medical images, receiving a prompt, the prompt being a request for a specific type of metadata to be inferred from the plurality of digital medical images, determining, using a trained foundation model, one or more feature descriptors from the plurality of digital medical images based on the prompt, and causing to, or providing for, output the one or more feature descriptors for each of the plurality of digital medical images.

It is to be understood that both the foregoing general description and the following detailed description are exemplary and explanatory only and are not restrictive of the disclosed embodiments, as claimed.

### BRIEF DESCRIPTION OF THE FIGURES

The accompanying drawings, which are incorporated in and constitute a part of this specification, illustrate various exemplary techniques and together with the description, serve to explain the principles of the disclosed techniques.

FIG. 1A depicts a block diagram of an exemplary system for generating and modifying foundation models, according to one or more techniques.

FIG. 1B depicts a block diagram of an exemplary system for generating foundation models, according to one or more techniques.

FIG. 1C depicts a block diagram of an exemplary system for modifying foundation models, according to one or more techniques.

FIG. 2 depicts an exemplary schematic for training a Masked AutoEncoder (MAE) model, according to one or more techniques.

FIG. 3 depicts an exemplary schematic for training a distilled MAE model, according to one or more techniques.

FIG. 4 depicts an exemplary schematic for training a hierarchical MAE model, according to one or more techniques.

FIG. 5 depicts an exemplary schematic for training a multi-modal model, according to one or more techniques.

FIG. 6 depicts an exemplary schematic for training a downstream task implementation, according to one or more techniques.

FIG. 7 depicts an exemplary method for processing digital medical images to infer metadata from those images, according to one or more techniques.

FIG. 8 depicts an exemplary method for processing digital medical images to infer at least one embedding from those images, according to one or more techniques.

FIG. 9 illustrates an example system or device that may execute techniques presented herein, according to one or more techniques.

### DETAILED DESCRIPTION

Reference will now be made in detail to the exemplary embodiments of the present disclosure, examples of which are illustrated in the accompanying drawings. Wherever possible, the same reference numbers will be used throughout the drawings to refer to the same or like parts.

The systems, devices, and methods disclosed herein are described in detail by way of examples and with reference to the figures. The examples discussed herein are examples only and are provided to assist in the explanation of the systems, devices, and methods described herein. None of the features or components shown in the drawings or discussed below should be taken as mandatory for any specific implementation of any of these systems, devices, or methods unless specifically designated as mandatory.

Also, for any methods described, regardless of whether the method is described in conjunction with a flow diagram, it should be understood that unless otherwise specified or required by context, any explicit or implicit ordering of steps performed in the execution of a method does not imply that those steps must be performed in the order presented but instead may be performed in a different order or in parallel.

As used herein, the term “exemplary” is used in the sense of “example,” rather than “ideal.” Moreover, the terms “a” and “an” herein do not denote a limitation of quantity, but rather denote the presence of one or more of the referenced items.

### Introduction

Techniques disclosed herein may describe systems and related methods for large-scale image processing using foundation models. Foundation models may include large-scale deep neural networks trained in a self-supervised manner and adaptable for downstream tasks. For example, millions of slides across hundreds of tissue types may be analyzed by a foundation model for universal whole slide representation for pattern discovery applied to cancer detection or segmentation, as well as one or more downstream prognostic clinical, or biomarker tasks.

This system and/or method may expose data in a manner that preserves differential privacy, allowing feature sharing for model development to occur without needing to employ federated learning strategies. Further, the system may be leveraged to analyze all biomarker signals across all tissue types to discover how multiple tests can be combined to more effectively predict disease, outcome, and/or treatment response.

### General System Overview

A general overview of the system described herein may include a foundation model trained on a large collection of images of pathology slides or other samples, covering a wide variety of organs, sampling types (i.e. biopsy, resection, aspiration, etc.), and the long tail of rare disease states and subtypes. The system may receive a plurality of medical images and a prompt to infer metadata from the plurality of medical images. The one or more medical images may comprise, but are not limited to, whole slide images (WSI), e.g., pathology WSI, radiology images, confocal microscopy, etc. The prompt may be a request for a specific type of metadata to be inferred from the image. The types of metadata may contain, but are not limited to, supplemental medical images from the case, structured diagnostic reports, unstructured free text reports, genomic data, proteomic data, treatment data, responses, diagnoses, etc.

Various further inputs may be received that may vary the output of the trained foundation model. In some techniques, the foundation model may receive one or more query constraints. Query constraints may include judgments or hypotheses from a clinician or expert, e.g., a clinician or expert that is using the system. Metadata estimations consistent with the one or more query constraints may be output by the foundation model if one or more query constraints are received. In some techniques, the trained foundation model may receive free text, such that the trained foundation model may output a structure synoptic diagnostic report based on the plurality of digital medical images and the free text query constraints typed by the users. The input free text content may include at least one of (1) specific histological details, (2) clinical context involving patient history and other modalities of tests, (3) diagnostic criteria, like applying WHO standard, etc., (4) information specific to the staining and markers, (5) morphological observations, (6) Instructions for Report Format, (7) concerns of the input data quality, (8) Comparative Analysis, (9) Special Instructions, etc. The output synoptic diagnostic report may be iteratively adjusted based on new input text prompts provided by the users.

In some techniques, the foundation model may run with a query. The foundation model may use one or more queries to request one modality be inferred from another. For example, a query may be used to request a diagnostic report from an image. In some techniques, the foundation model may run without a query. The foundation model may produce a general feature descriptor that may be used to train downstream models on more specialized tasks.

In some techniques, a content-based retrieval system may be used to determine a collection of related digital medical images or cases based on the metadata associated with each of the digital medical images or cases. The content-based retrieval system may receive content-based constraints to modify queries for content retrieval. The content-based constraints may include instructions to include or exclude specific types of metadata or attributes of the metadata.

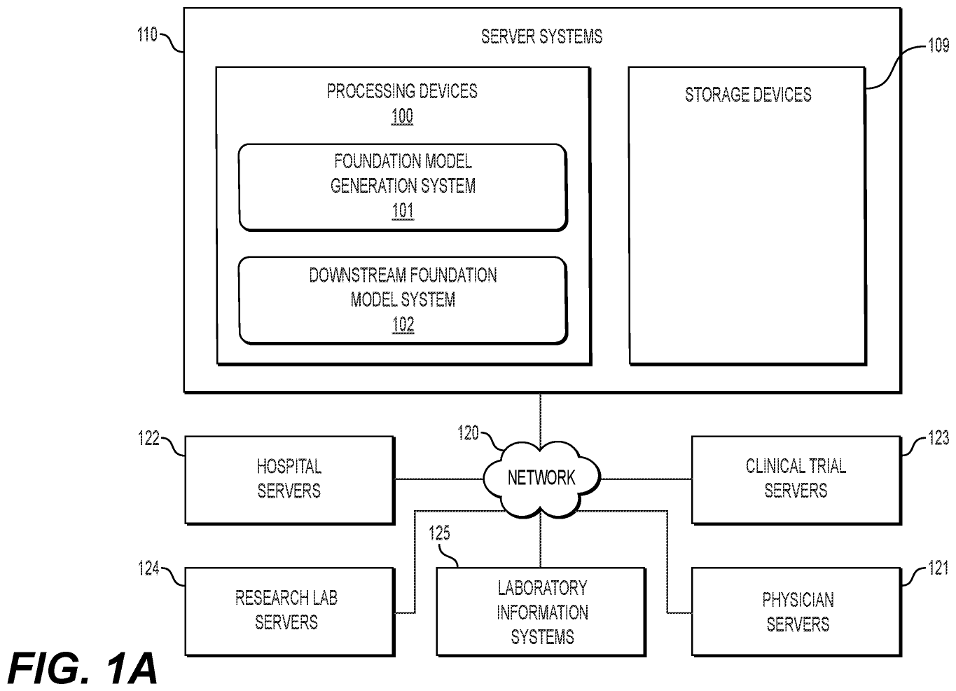

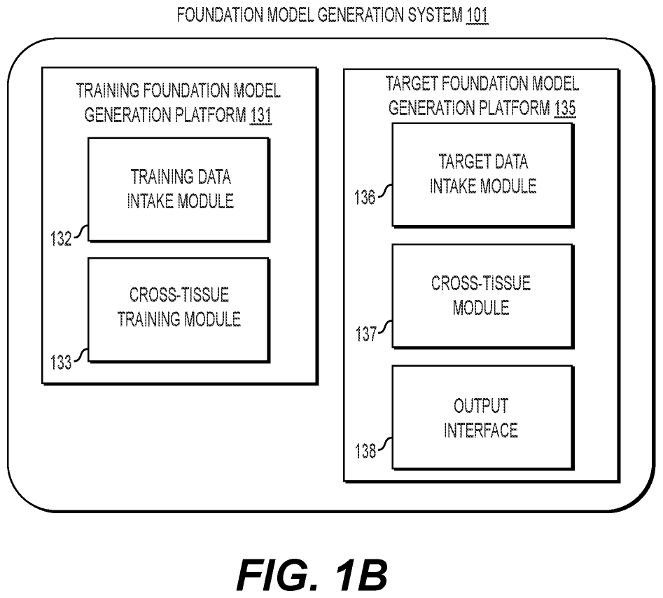

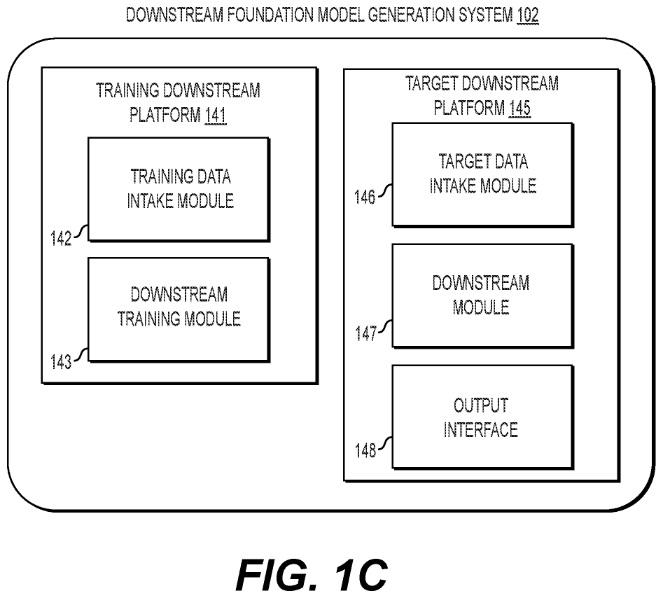

FIG. 1A depicts an exemplary system for processing digital medical images to infer metadata from those images, according to one or more techniques. Illustrated in FIG. 1A is an electronic network 120 that may be connected to physician servers 121, hospital servers 122, clinical trial servers 123, research lab servers 124, and/or laboratory information systems 125, for example, through one or more computers, servers, and/or handheld mobile devices. According to an exemplary aspect of the present disclosure, network 120 may be connected to server systems 110, which may include one or more processing devices 100, e.g., configured to run or execute foundation model generation system 101, and/or storage devices 109. Foundation model generation system 101 may be configured for processing digital medical images to generate a foundation model. Downstream foundation model system 102 may be configured for modifying a foundation model (e.g., a foundation model generated by foundation model generation system 101 or another system) for at least one downstream use, according to exemplary aspects of the present disclosure. While foundation model generation system 101 and downstream foundation model system 102 are depicted as separate systems in FIG. 1A, it should be understood that, in other examples, foundation model generation system 101 and downstream foundation model system 102 may be sub-systems of a larger system.

Physician servers 121, hospital servers 122, clinical trial servers 123, research lab servers 124, and/or laboratory information systems 125 may create or otherwise obtain data, such as pathology slides, digital medical images, clinical reports, free-text reports, immunohistochemistry (IHC) or immunofluorescent slides, Computed Tomography (CT) scans, genomic data (e.g., gene expression data, genomic variants, etc.), proteomic data, clinical data, etc. For example, the pathology slides may include a wide variety of organs, sampling types (e.g., biopsy, resection, aspiration, etc.), disease states, and/or disease subtypes. In another example, the digital medical images may include digital pathology images, including one or more patients' whole slide image(s), cytology specimen(s), histopathology specimen(s), slide(s) of the cytology specimen(s), digitized images of the slide(s) of the histopathology specimen(s), or any combination thereof, that may be created or obtained. Additionally or alternatively, the digital medical images may include images of other modality types, including digital multiplex immunofluorescent images, digital multiplex immunohistochemistry images, magnetic resonance imaging (MRI), computed tomography (CT), X-ray, nuclear medicine imaging, or ultrasound, that may be created or obtained.

Expression data may include patient-specific or non-patient-specific tumor sequencing data, protein expression levels, and/or non-coding RNA expression levels. Expression data may be utilized by both medical professionals (e.g., pathologists, physicians, etc.) and AI systems alike for training purposes to improve accuracy in generating and/or modifying a foundation model, among other tasks. A greater availability of expression data presenting a particular condition or disease enhances both medical professionals' and AI systems' ability to learn given the increased variability in the presentation among the expression data. However, large amounts of expression data remain unavailable for individual genetic mutations in each tumor type, which necessarily limits an amount of variability that can be learned. For example, treatment of a patient-specific tumor may be made difficult due to genotype variance compared to another patient with the same phenotype but a different genotype.

Genomic variants may include mutations in individual genes of a given gene complex or signaling pathway, such as the SWItch/Sucrose Non-Fermentable (SWI/SNF) complex (e.g., ARIDIA, ARID1B, ARID2, PBRM1, SMARCA4 and SMARCB1) or the Receptor Tyrosine Kinase (RTK)/Ras/MAP kinase (MAPK) pathway (e.g., ERBB2, ERBB3, ERBB4, SOS1, HRAS, BRAF, MAP2K1, and MAPK1), etc. Clinical data may include age, medical history, cancer treatment history, family history, past biopsy or cytology information, tumor sequencing information, messenger ribonucleic acid (mRNA) expression levels, gene network graphs (pre-treatment and/or post-treatment), overall survival data, progression-free survival with corresponding censored data, 5-year survival rates, drug treatment outcome data, etc.

Data discussed herein may be communicated between server systems 110 and physician servers 121, hospital servers 122, clinical trial servers 123, research lab servers 124, and/or laboratory information systems 125 over network 120 in a digital and/or electronic format.

Server systems 110 may include one or more storage devices 109 for storing data, e.g., data received from at least one of physician servers 121, hospital servers 122, clinical trial servers 123, research lab servers 124, and/or laboratory information systems 125. For example, the foundation model generated by foundation model generation system 101 may be stored within the one or more data stores, e.g., storage devices 109.

Server systems 110 may include processing devices 100 for processing the data stored in storage devices 109. Server systems 110 may include one or more machine learning tool(s) or capabilities. For example, processing devices 100 may execute one or more machine learning systems utilized by foundation model generation system 101 and/or downstream foundation model system 102, according to one or more techniques. In some examples, outputs of the machine learning systems may be stored in storage devices 109 for use by other systems or processes, as described in detail below. Alternatively or in addition, the present disclosure (or portions of the system and methods of the present disclosure) may be performed on a local processing device (e.g., a laptop).

According to an exemplary aspect of the present disclosure, foundation model generation system 101 may be configured to generate a foundation model. The foundation model may be generated with a large number (e.g., hundreds, thousands, millions, etc.) of slides across numerous (e.g., tens, hundreds, thousands, etc.) tissue types, e.g., without annotations. According to an exemplary aspect of the present disclosure, downstream foundation model system 102 may be configured to modify at least one foundation model for downstream tasks, uses, etc. The techniques discussed herein may work across different tissues and/or abnormalities and tasks, e.g., with small sample size, operate in a pan-cancer and/or pan-tissue manner, enable improved generalization to new domains (e.g., other scanners or hospitals), etc.

FIG. 1B depicts an exemplary system (e.g., foundation model generation system 101) for generating a foundation model, according to an exemplary aspect of the present disclosure. The foundation model generation system 101 may include a training foundation model generation platform 131 and/or a target foundation model generation platform 135.

The training foundation model generation platform 131, according to one technique, may create or receive one or more data of foundation model training data used to generate and train one or more machine learning models that, when implemented, generate a foundation model. According to one technique, the training foundation model generation platform 131 may include a plurality of software modules, including a training data intake module 132 and a cross-tissue training population module 133. The data and/or machine learning systems output by training foundation model generation platform 131 may be stored, e.g., in storage device 109, or used by other systems, e.g., target foundation model generation platform 135.

Training data intake module 132, according to one aspect, may create or receive foundation model training data that may be used to generate at least one foundation model. The foundation model training data may be received from any one or any combination of server systems 110, physician servers 121, hospital servers 122, clinical trial servers 123, research lab servers 124, and/or laboratory information systems 125. Foundation model training data may come from real sources (e.g., humans, animals, etc.) or may come from synthetic sources (e.g., graphics simulators, graphics rendering engines, 3D models, etc.).

The foundation model training data may include one or more data corresponding to pathology slides, digital medical images, clinical reports, free-text reports, IHC or immunofluorescent slides, CT scans, genomic data, proteomic data, clinical data. In some examples, a subset of foundation model training data may overlap between or among the various data for pathology slides, digital medical images, clinical reports, free-text reports, IHC or immunofluorescent slides, CT scans, genomic data, proteomic data, clinical data. The foundation model training data may be stored on a digital storage device, e.g., one of storages devices 109.

The cross-tissue training module 133 may be configured to generate a trained foundation model based on the foundation model training data. As discussed in further detail herein, cross-tissue training module 133 may be configured to train the foundational model using any suitable technique(s), e.g., multi-modal, without annotations, etc. In one technique, cross-tissue training module 133 may be trained to learn at least one relationship between modalities (e.g., between clinical reports, free-text reports, IHC or immunofluorescent slides, CT scans, genomic data, proteomic data, etc.), and/or draw inferences between the various modalities. The foundation model training data may be received by cross-tissue training module 133 from any one or any combination of server systems 110, physician servers 121, hospital servers 122, clinical trial servers 123, research lab servers 124, laboratory information systems 125, and/or training data intake module 132. Cross-tissue training module 133 may output, e.g., a trained foundation model, that may be stored, e.g., in storage devices 109, and/or utilized by target foundation model generation platform 135.

According to one technique, the target foundation model generation platform 135 may include software modules, such as a target data intake module 136, a cross-tissue module 137, and an output interface 138. Target foundation model generation platform 135, according to one aspect, may receive a request, e.g., to create image features that represent various tissue morphologies and architectures for both healthy and disease conditions. As discussed in more detail below (e.g., see FIG. 1C), these features may be used to train downstream foundation models for a wide variety of tasks such as cancer detection, segmentation, biomarker identification, etc. Target foundation model generation platform 135 may be configured to execute one or more of the foundation models trained by training foundation model generation platform 131. For example, target foundation model generation platform 135 may further train a foundation model using embeddings (e.g., embeddings generated by the trained foundation model) to generate an image analysis model. In some techniques, the request may be received from any one or any combination of the physician servers 121, hospital servers 122, clinical trial servers 123, research lab servers 124, and/or laboratory information systems 125. In some techniques, the request may be automatically received from downstream foundation model system 102 in response to downstream foundation model system 102 receiving a request to generate, train, etc., a foundation model and/or modify a trained foundation model.

Target data intake module 136, according to one aspect, may create or receive the target data that may be used as an input for one or trained more machine learning systems to modify a foundation model. For example, target data intake module 136 may receive digital medical images, which may be used as an input for one or more trained foundation models. The target data may be received from any one or any combination of server systems 110, physician servers 121, hospital servers 122, clinical trial servers 123, research lab servers 124, and/or laboratory information systems 125. Target data may come from real sources (e.g., humans, animals, etc.) or may come from synthetic sources (e.g., graphics simulators, graphics rendering engines, 3D models, etc.). Target data intake module 136 may create or receive the target data. Target data may include at least one of digital medical images, clinical reports, free-text reports, IHC and/or immunofluorescent slides, CT scans, genomic data, proteomic data, other medical data, etc. In some examples, a subset of target data may overlap between or among the various data for images and/or clinical data. The target data may be stored on a digital storage device, e.g., one of storages devices 109.

Cross-tissue module 137 may include any suitable foundation model machine learning systems, including but not limited to, graph neural networks, convolutional neural networks, transformer neural networks, etc. Cross-tissue module 137 may execute the various foundation models, such as the foundation model generated by training graph generation platform 131. Cross-tissue module 137 may determine at least one relationship between modalities (e.g., between clinical reports, free-text reports, IHC or immunofluorescent slides, CT scans, genomic data, proteomic data, etc.), and/or draw inferences between the various modalities. For example, cross-tissue module 137 may synthesize a natural language description or structured diagnostic report from a Hematoxylin and Eosin (H&E) slide, render a synthetic IHC based on the H&E, and/or predict a full genomic panel. In another example, the model may produce representations for cells, localized regions (e.g., patches), a whole slide image, and/or for a group of slides, etc.

The output interface 138 may be used to output the trained foundation model (e.g., generated by training foundation model generation platform 131) and/or the modified foundation model (e.g., modified by target foundation model generation platform 135) (e.g., to a screen, monitor, storage device, web browser, etc.). According to some techniques, output interface 138 may output the trained foundation model to downstream foundation model system 102 for use as input in a subsequent process described below. Foundation models and other data produced or used by foundation model generation system 101 may be stored in one or storage devices 109.

FIG. 1C depicts an exemplary system (e.g., downstream foundation model system 102) for modifying at least one foundation model, e.g., for downstream tasks, according to an exemplary technique of the present disclosure. Downstream foundation model system 102 may include a training downstream platform 141 and/or a target downstream platform 145.

According to one technique, the training downstream platform 141 may include software modules, such as a training data intake module 142 and a downstream training module 147. Training data intake module 142, according to one aspect, may create or receive training data (e.g., foundation model training data) that may be used to train one or more machine learning systems to modifying at least one foundation model for downstream tasks. For example, downstream training module 143 may further train a foundation model on embeddings (e.g., embeddings generated by the trained foundation model) to generate an image analysis model. The training data may be received from any one or any combination of server systems 110, physician servers 121, hospital servers 122, clinical trial servers 123, research lab servers 124, and/or laboratory information systems 125. Training data may come from real sources (e.g., humans, animals, etc.) or may come from synthetic sources (e.g., graphics simulators, graphics rendering engines, 3D models, etc.). The training data intake module 142 may create or receive the downstream training data. For example, the downstream training data may include embeddings generated by the foundation model (e.g., by cross-tissue module 137), such as cancer and/or biomarker detection, cancer and/or biomarker scoring, multi-model diagnoses, prognoses, treatment planning, content-based retrieval, etc. In some examples, a subset of downstream training data may overlap between or among the various downstream training data.

In some examples, the training data may be a direct output of one or more of the machine learning systems (e.g., foundation models). In other examples, the output of one or more of the machine learning systems may be used as input to further processes that enable modification of a foundation model. The downstream training datasets may be stored on a digital storage device, e.g., one of storages devices 109.

The downstream training module 143 may generate, using the downstream training data as input, one or more modified foundation model, e.g., to be used for a downstream purpose. In some examples, a third party may generate the one or more trained machine learning systems and provide the trained machine learning system(s) to server systems 110 for storage (e.g., in storage devices 109) and/or execution by downstream foundation model system 102. Downstream training module 143 may train a transformer, a graph neural network, or any other suitable type of machine learning system to modify (e.g., further train) a foundation model (e.g., obtained from foundation model generation system 101) for a given downstream use. Training prediction module 143 may store the modified foundation model in a database, e.g., storage devices 109, along with other foundation models, e.g., foundation models and modified foundation models.

According to one technique, the target downstream platform 145 may include software modules, such as a target data intake module 146, a downstream module 147, and an output interface 148. Target data intake module 146 may receive one or more target inputs, including, but not limited to, a modified foundation model, embeddings generated by a trained foundation model (e.g., modality-specific embeddings), etc. For example, the target data may be received from any one or any combination of the server systems 110, physician servers 121, hospital servers 122, clinical trial servers 123, research lab servers 124, and/or laboratory information systems 125.

Target data intake module 146 may provide the one or more inputs to the downstream module 147 to generate an output via the modified foundation model. Downstream module 147 may execute the various modified foundation models generated by training downstream platform 141 to generate at least one output for a downstream task.

Downstream module 147, according to one aspect, may receive a request to execute one or more of the machine learning systems trained by training downstream platform 141 (e.g., a modified foundation model) to predict an output for at least one downstream task. For example, the request may be received from any one or any combination of the server systems 110, physician servers 121, hospital servers 122, clinical trial servers 123, research lab servers 124, and/or laboratory information systems 125. In another example, the request may be automatically generated by downstream foundation model system 102 in response to detecting an output from another system, e.g., from foundation model generation system 101. In some implementations, downstream module 147 may be configured to automatically predict an output for at least one downstream task, e.g., based on the input target data and/or the modified foundation model.

The output interface 148 may be used to output the predicted output for the at least one downstream task (e.g., to a screen, monitor, storage device, web browser, etc.).

### Exemplary Techniques

The foundation model (e.g., trained by training foundation model generation platform 131) may be trained in a self-supervised manner using at least the plurality of medical images. Exemplary training methods may include Masked Autoencoder (MAE) training, Distilled MAE training, Hierarchical MAE training, Hierarchical Distilled MAE training, contrastive methods, Multi-Modal Training, etc.

### Masked Autoencoder (MAE) Model

An MAE foundation model may follow an autoencoding scheme that reconstructs the original data given partially masked input data. The MAE foundation model may have an encoder, e.g., a Vision Transformer (ViT) Encoder that maps observed data to a latent space, and a decoder, e.g., a ViT Decoder, that reconstructs original data from the latent space. The MAE foundation model may operate based on an asymmetric design that allows the encoder to operate on partial, observed data that has no mask tokens, and allows the decoder to reconstruct the full data from the latent space and mask tokens.

### MAE Training

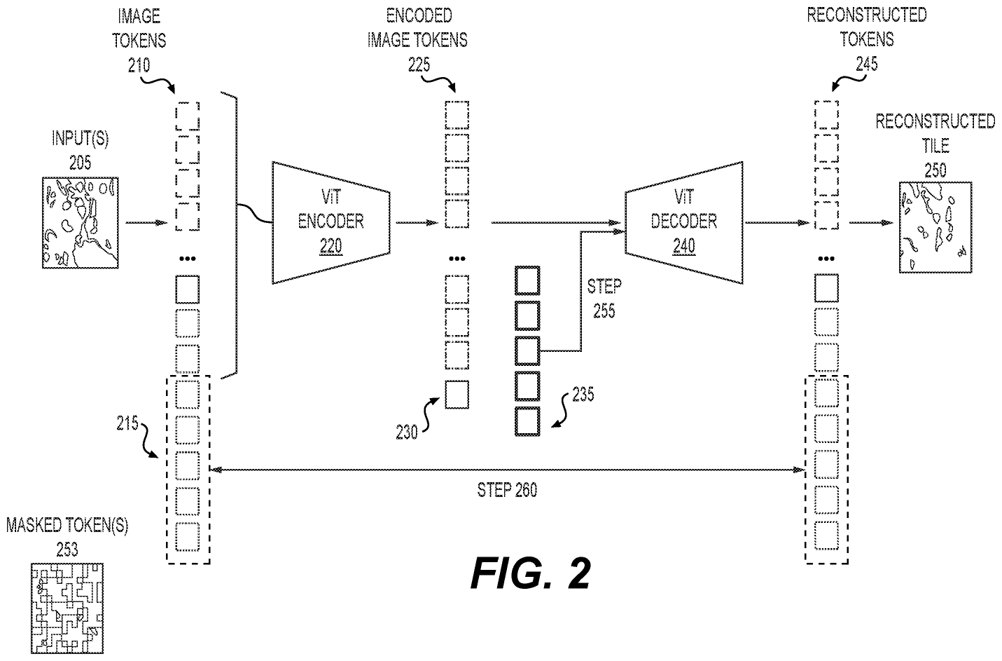

As depicted in FIG. 2, an exemplary method for training the MAE model may include receiving a plurality of inputs 205, such as medical images, one or more prompts, etc. As depicted in FIG. 2, the plurality of medical images may be divided into a plurality of fixed-size patches (image tokens 210). A subset of the image tokens 215 may be intentionally removed from the plurality of medical images, leaving a remaining plurality of image tokens.

An encoder, e.g., ViT Encoder 220, may output one or more encoded image tokens 225 based on the remaining plurality of image tokens. In some techniques, a classification token 230 may be added at the start of the encoded image tokens 225. The classification token 230 may be a network-specific vector of numbers and may be used for summarizing the image tile representation. In some techniques, masked tokens 235 may be appended with position encoding applied to each respective encoded image token.

The masked tokens 235, and optional classification token 230, may be fed into a ViT Decoder 240. The VIT Decoder 240 may be used to reconstruct the original image tokens (e.g., image tokens 210) to match or substantially match the original image pixel values. ViT Decoder 240 may generate at least one output, e.g., reconstructed tokens 245, reconstructed tile 250, etc. In some techniques, the network may be optimized using L2 image reconstruction loss applied on the removed vision tokens (e.g., masked token(s) 253) only (e.g., see step 255 of FIG. 2). In some techniques, VIT Encoder 220 may be trained to align a subset of the image tokens 215 with reconstructed tokens 245 and may force the network to learn global structure (see step 260).

### Distilled MAE Model

A distilled MAE model, similar to a MAE model, may include an encoder and a decoder (e.g., ViT decoder 321). The encoder of the distilled MAE model further includes a student encoder (e.g., a student ViT encoder) and a teacher encoder (e.g., a teacher ViT encoder). The distilled MAE model may have any of the features or training steps discussed in relation to any of the other models described herein, unless otherwise specified.

### Distilled MAE Training

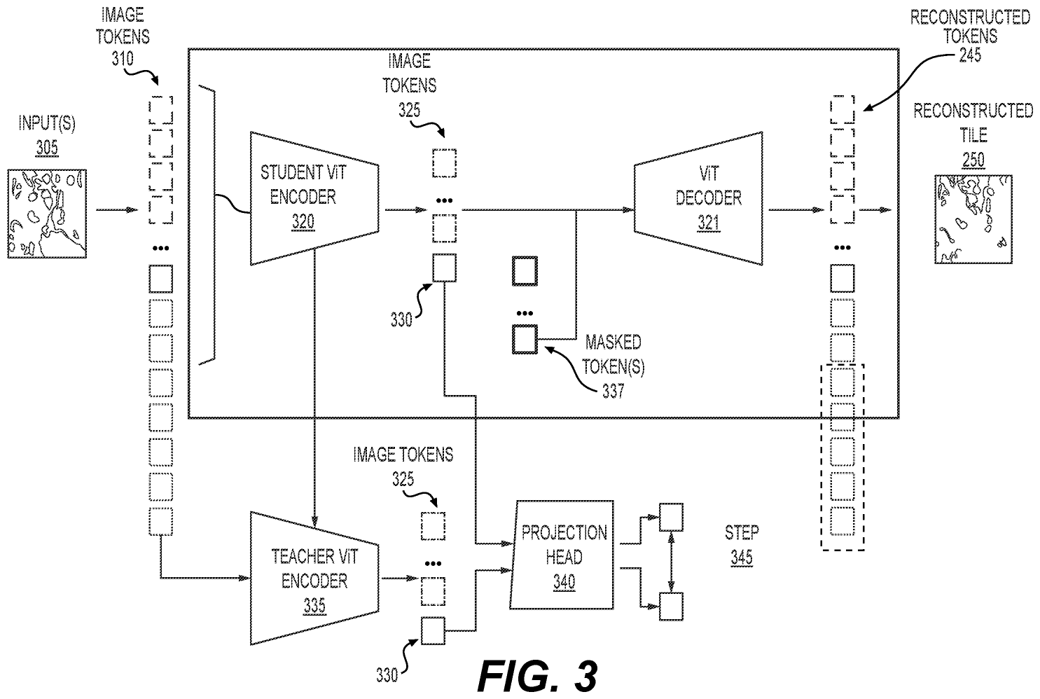

An exemplary method for training the distilled MAE model is described in FIG. 3. The distilled MAE model may receive a plurality of input 305, such as medical images, one or more prompts, etc. For each of the plurality of inputs 305, a Hue, Saturation, and Value (HSV)-based foreground detection algorithm (not depicted) may extract image tokens 310 of certain sizes, e.g., 224×224, on the tissues of interests. The glass regions may be discarded. During training, inputs 305 may be randomly sampled from the entire database with balanced metadata sampling. As depicted in FIG. 3, a student encoder, e.g., Student VIT Encoder 320, may break the plurality of inputs 305 (e.g., plurality of medical images) into a plurality of fixed-size patches (image tokens 325). A subset of the image tokens 325 may be intentionally removed from the plurality of medical images, the Student ViT Encoder 320 outputting one or more masked tokens 337.

Since the classification token 330 may not be explicitly trained, a multilayer perceptron (MLP) may be applied similarly to self-distillation with no labels (DINO) on top of the classification token 330 to project the tile embeddings. A Student ViT Encoder 320 with masked tokens 337 may be trained with a distilled loss to predict the classification tokens 330 predicted by a Teacher ViT Encoder 335 fed with all the image tokens 310 without masking. The Teacher ViT Encoder 335 may be updated using the moving average of the Student ViT Encoder 320 at the end of each training batch. The distilled loss may be combined with the image reconstruction loss using a weighted sum to encourage the network to learn both the local scale patterns while being capable of summarizing the image tile content. A projection head 340 may convert the classification token 330 from one 1-dimensional vector to another 1-dimensional vector of a different size, which may train the Student ViT Encoder 320 to align with the distribution from the Teacher ViT Encoder 335 and may force the network to learn global structure (see step 345).

### Hierarchical MAE Model

A hierarchical MAE model may include a tile-level ViT Encoder feeding into either of a MAE or a distilled MAE. A first set of tiles may be fed into a tile-level (e.g., pre-trained) ViT Encoder to output class tokens. The class tokens may then be fed into either a MAE or a distilled MAE to output reconstructed tokens.

### DINOv2 Model

The DINOv2 foundation model may implement a novel self-supervised learning architecture, utilizing a self-distillation mechanism. Central to the DINOv2 foundation model are two Vision Transformers (ViT): a student network and a teacher network. These networks may process augmented views of the same image in parallel, with the student network tasked to predict the teacher network's output. The teacher network may be distinctively updated as an exponential moving average of the student network's weights. This architecture may allow DINOv2 to learn by maintaining consistency between different image views, eliminating the need for labeled data in its training process.

### DINOv2 Training

DINOv2 Training may be similar to MAE training (discussed in more detail below), which may include a diverse array of medical images. Each image may be subjected to a set of augmentations to create multiple and/or varied views. These augmented views may be split into a series of fixed-size patches and/or may be analogous to image tokens in the MAE method. The student network may receive a subset of these patches, while the teacher network may receive either the same or a different subset, which may include patches that may be withheld from the student network to create a knowledge gap.

The student network, e.g., equipped with a Vision Transformer (ViT) architecture, may process the patches to produce a set of encoded representations. The teacher network, operating in a non-gradient-following manner, may update its weights as an exponential moving average of the student's parameters over time. This setup may generate a continuously evolving target for the student network, which may drive it to learn richer representations as it attempts to predict the teacher's outputs.

During training, both networks may utilize self-attention mechanisms inherent to ViTs, which may enable them to focus on informative parts of the input data. The student network's predictions may be compared to the teacher network's outputs, and the discrepancy between them may be minimized using a distillation loss function. This loss may be calculated solely based on the prediction of the encoded representations, which may encourage the student to emulate the teacher's behavior. As training progresses, the student network may be encouraged to learn both local and global structures within the data, which may promote a comprehensive understanding of the visual content without reliance on manual annotations or labels.

### Hierarchical MAE (or Slide-Level Foundation Model) Training

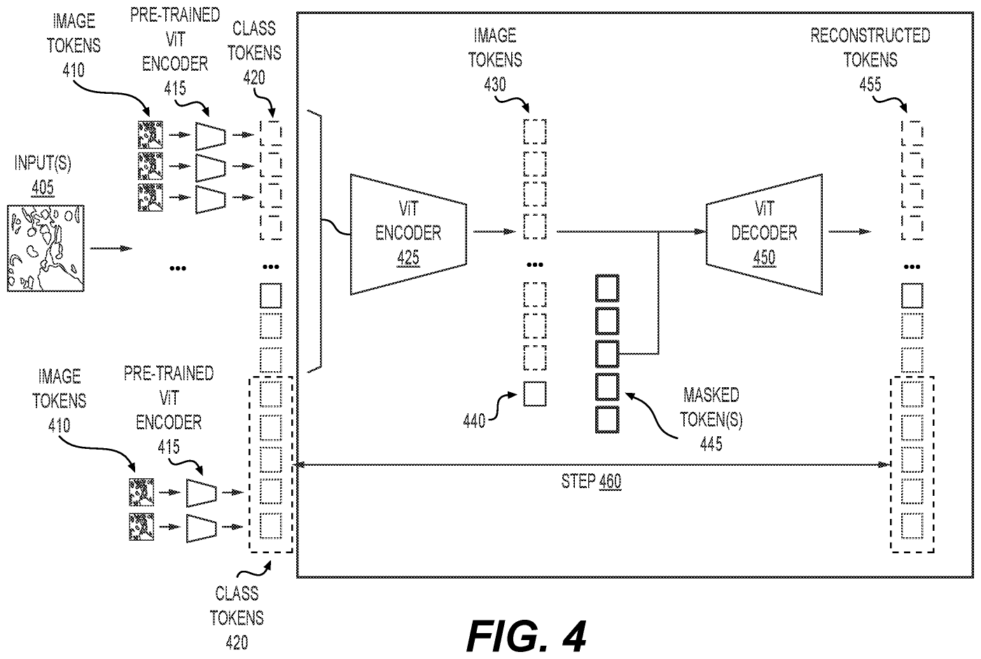

An exemplary method for training the hierarchical foundation model is depicted below in FIG. 4. As depicted, a tile-level ViT Encoder (e.g., Pre-Trained ViT Encoder 415) may be trained to output tile-level class tokens 420 based on a plurality of inputs 405 (e.g., image tiles), e.g., using methods described herein. The tile-level ViT Encoder (e.g., Pre-Trained VIT Encoder 415) may be trained using either of MAE training or distilled training, as described herein. Once the tile-level ViT Encoder is trained, the predicted tile-level class tokens 420 may be grouped into a grid of tiles, e.g., 16×16, and fed into a grid-level ViT Encoder (e.g., ViT Encoder 425), as discussed above. The grid-level ViT Encoder may have similar architecture to the tile-level ViT Encoder to represent the spatial organization of the tiles, similar to Hierarchical Image Pyramid Transformer (HIPT). The same distilled MAE combined loss may be applied to the class tokens 420, instead of the image tokens 430.

The masked tokens 445, and optional classification token 440, may be fed into a ViT Decoder 450. The ViT Decoder 450 may be used to reconstruct the original image tokens (e.g., image tokens 410) to match or substantially match the original image pixel values. ViT Decoder 450 may generate at least one output, e.g., reconstructed tokens 455, a reconstructed tile/image, etc. In some techniques, VIT Encoder 425 may be trained to align a subset of the class tokens 420 with reconstructed tokens 455 and may force the network to learn global structure (see step 460).

The grid-level class-tokens from the same slide can be aggregated using an aggregator network (not depicted) to obtain the representation of the entire whole slide. The aggregator network can be a similar ViT network or a conventional MLP/long short-term memory (LSTM)/Convolutional Neural Network (CNN) network. The slide-level aggregator network may be pre-trained using at least one of three key methodologies: (1) Self-Supervised Learning, (2) Image-Text Pretraining, and/or (3) Supervised Training with Slide or Group Level Ground Truth Labels.

Self-Supervised Learning may involve training the aggregator network to understand and interpret complex patterns within the slide images without explicit labeling. By analyzing the inherent structures and features of the slides, the model may learn to discern subtle nuances that are critical for accurate interpretation in digital pathology. This self-learning approach may facilitate a deeper understanding of the slide images, laying a solid foundation for further, more specialized training.

Following the principles of models like Contrastive Language-Image Pretraining (CLIP) or Contrastive Captioners are Image-Text Foundation Models (COCA), the aggregator network may undergo an Image-Text Pretraining phase, where it may learn to correlate textual descriptions, such as diagnosis reports, molecular test reports, etc. with corresponding slide images. This training phase may enable the model to recognize visual features and/or understand contextual information presented in text form. Such dual-modality training may enhance the model's capability to process and interpret complex medical images in conjunction with relevant textual data, such as clinical notes or research findings.

To refine the model's accuracy and adapt it to specific requirements of digital pathology, Supervised Training with Slide or Group Level Ground Truth Labels may be employed. In this phase, ground truth labels for slides or groups of slides may be used. These labels may be extracted automatically using advanced Large Language Models (LLMs), which may analyze textual data associated with the slides to generate precise and/or contextually relevant labels. The LLM may be provided detailed system prompts containing detailed description of the groundtruth labels and/or the expected format to be extracted. The ground truth labels may be obtained by feeding the textual reports to the LLM agents and/or parsing the textural responses using heuristic pipelines. This approach may train the model on visual data and/or on substantially accurately labeled datasets, which may enhance its predictive accuracy and/or reliability.

### Multi-Modal Model

The foundation model may also be a multi-modal model and/or trained in a multi-modal fashion, incorporating additional associated data such as clinical reports, free-text reports, hematoxylin and eosin (H&E) stains, immunohistochemistry (IHC) slides, immunofluorescent slides, CT scans, genomic data, proteomic data, etc. A model trained in this manner may learn the relationships between the various modalities, which may allow inferences to be drawn between them. For example, the model could synthesize a natural language description or structured diagnostic report from an H&E slide, render a synthetic IHC based on the H&E, or predict a full genomic panel. The model may also produce representations for cells, localized regions (e.g., patches), a WSI, for a group of slides, etc.

### Multi-Modal Training

An exemplary method for training the multi-modal model is described as follows. The multi-modal model may conduct a search to find slides with similar characteristics. For example, if trained to map WSI to proteomes, the multi-modal model may produce the proteome for a given patient given only the WSI, or use a proteome to find WSI that are similar to that proteome. Multi-modal inputs may have an embedding for each modality, and then may use the combination of modalities for downstream tasks, e.g., treatment recommendation.

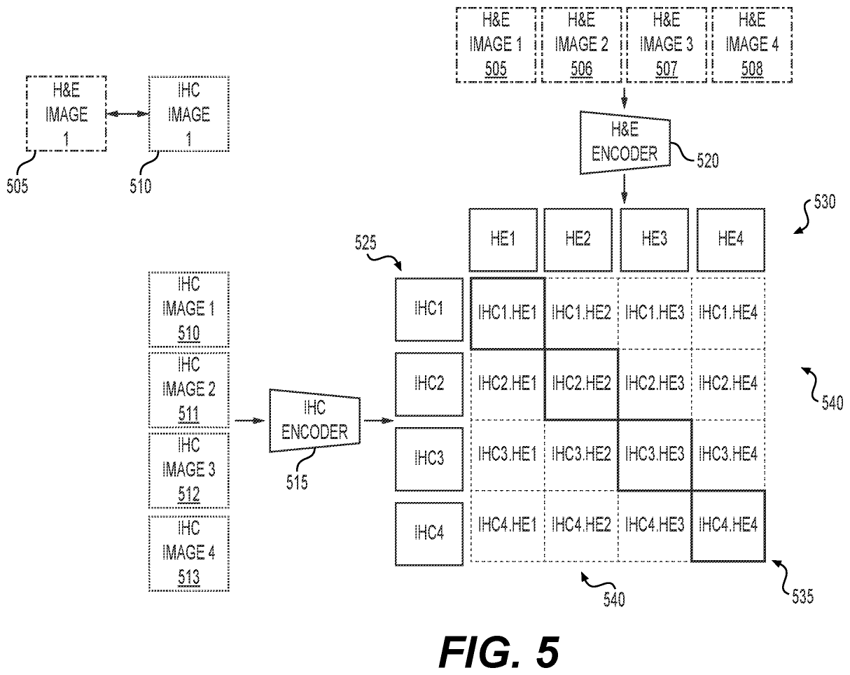

As depicted in FIG. 5, the multi-modal model may be trained to analyze data based on modalities. In an example where the multi-modal model may analyze IHC and H&E images, the multi-modal model may be trained to match H&E Image 1 (505) and IHC Image 1 (510) based on a shared tissue type, H&E Image 2 (506) and IHC Image 2 (511) based on a shared tissue type, H&E Image 3 (507) and IHC Image 3 (512) based on a shared tissue type, H&E Image 4 (508) and IHC Image 4 (513) based on a shared tissue type, etc.

The respective data may be input to modality-specific encoders. For example, the H&E images (e.g., H&E images 505, 506, 507, 508, etc.) may be input to an H&E Encoder 520, and the IHC images (e.g., IHC images 510, 511, 512, 513, etc.) may be input to an IHC Encoder 515. The modality-specific encoders may output encodings, e.g., H&E encodings 530 and IHC encodings 525. The encodings may be used to learn the relationships between the various modalities. In some techniques, the multi-modal model may be trained to maximize diagonal entries 535 while minimizing off-diagonal entries 540 of the encodings.

Training the multi-modal model and the modality-specific encoders may occur contemporaneously, or the multi-modal model may be trained after the modality-specific encoders.

### Downstream Task Implementation

Generally, a foundation model may be adapted to a wide range of downstream tasks, enabling greatly increased capabilities, such as cancer detection, segmentation, biomarker identification, or morphology-based image retrieval. The foundation model may be further optimized or fine-tuned for downstream tasks independently or jointly. For example, image analysis models can be trained on the embeddings generated by the foundation model, including cancer or biomarker detection, cancer or biomarker scoring, multi-model diagnosis, prognosis, treatment planning, content-based retrieval, etc.

In one technique, a multi-modal model may be configured to generate an embedding for each modality, the combination of modalities then being configured for downstream tasks, e.g., generating a treatment recommendation. In another technique, the foundation model may be configured for biomarker-related tasks. For example, given a WSI, a downstream task implementation may be configured to detect the presence or score of biomarkers, such as human epidermal growth factor receptor 2 (HER2), Kirsten rat sarcoma virus (KRAS), microsatellite instability (MSI), epidermal growth factor receptor (EGFR), etc. In another technique, the foundation model may be configured for clinical-related tasks. For example, given a WSI, a downstream task implementation may be configured to detect the presence, subtype, grade, origin site of a cancer in the WSI. In another technique, the foundation model may be configured for survival/outcome-related tasks. For example, given a WSI, a downstream task implementation may be configured to predict the overall survival, disease-free survival, and/or prognosis-free survival of the patient. In another technique, the foundation model may be configured for treatment outcome-related tasks. For example, given a WSI, a downstream task implementation may be configured to predict the immuo-oncological response to a particular treatment. In any of the above examples, given an amount of WSI image-label pairs, a second model may be trained on top of the outputs of the foundation model using the WSI image-label pairs.

In some techniques, downstream task implementation may include determining output targets that were not contained within metadata types based on one or more feature descriptors for each of the plurality of digital medical images. The output targets may include any combination of learning markers for drug response, building a model to replicate an existing biomarker, learning a novel biomarker from test data or other ground-truth indicators, or predicting additional disease states or diagnostics.

The implementation of a content-based retrieval system using embeddings from a digital pathology foundation model may involve several key steps to optimize performance and storage efficiency. In a first step, the embeddings of image tiles, which may be derived from Whole Slide Images (WSIs), may be stored in a vector database. This database may be designed for fast querying, and/or may enable efficient retrieval of similar image tiles based on their embeddings. To optimize storage, the system may identify and/or save only a few representative tiles from each WSI. This approach may reduce the storage footprint while maintaining the ability to retrieve a comprehensive range of similar tiles.

When a user selects a region of interest (ROI) on a WSI, the system may process this ROI by extracting the embeddings of the tiles enclosed within it. Each of these embeddings may serve as an individual query to the vector database. The system may retrieve slides and/or ROIs from the database, e.g., that have similar embeddings to those of the query tiles. To rank the returned slides and ROIs, the system may calculate similarity scores. These scores may measure how closely the tiles found in a retrieved slide match the bag of query tiles from the user's selected ROI. The system may support retrieval of most similar tiles and/or most dissimilar tiles by manipulating the similarity statistic.

This method may enable users to retrieve the most relevant WSIs and/or regions thereof, e.g., based on their specific query. The flexibility of the system may enable users to select ROIs of any shape, size, and/or magnification, which may ensure a highly customizable search experience. The foundation model's embedding-based approach may be applicable to various medical image types, e.g., H&E, IHC, and CT images, and may be configured to handle data sources ranging from individual WSIs to large, cross-institutional data lakes of WSI. This versatility may enhance the utility of the retrieval system, and/or may make it an invaluable tool in digital pathology for tasks such as contextualizing downstream task predictions with relevant text and image references. Additionally, it may provide an interface for domain experts to study the learned information stored within the foundation model's embeddings, which may allow for the exploration of new research avenues and formulation of novel downstream tasks.

Downstream task implementation may lower artificial intelligence development time and cost, may improve performance, and may enable the development of new products, especially on tasks with insufficiently labeled data on which to train deep neural networks. Further, embedding generation may be used for searching data lakes of WSI, improving search times and use of data stored within data lakes.

### Downstream Task Implementation Training

An exemplary method for training a downstream task implementation (e.g., modifying a trained foundation model) is described as follows.

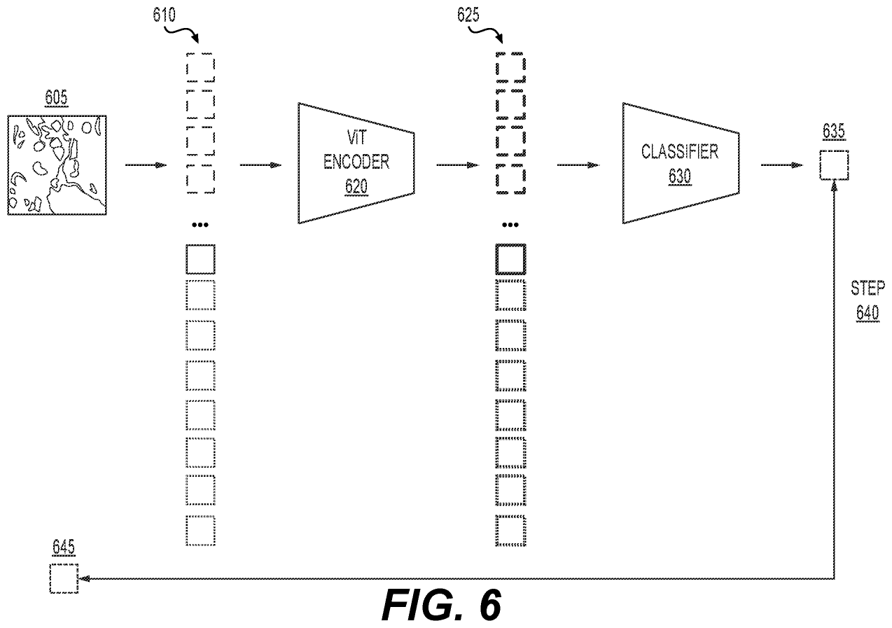

As depicted in FIG. 6, input(s) 605, e.g., image tiles, may be broken into image tokens 610 using methods described herein. The image tokens 610 may be inputted to a ViT Encoder 620, which may output encoded image tokens 625 using methods described herein. In some implementations, the ViT Encoder 620 may be a frozen or lower right (LR) encoder. The encodings may be input to a classifier 630, which may be trained to classify the encodings (e.g., encoded image tokens 625) to output a predicted label 635. The predicted label may be trained to align with a ground truth label 645 (step 640).

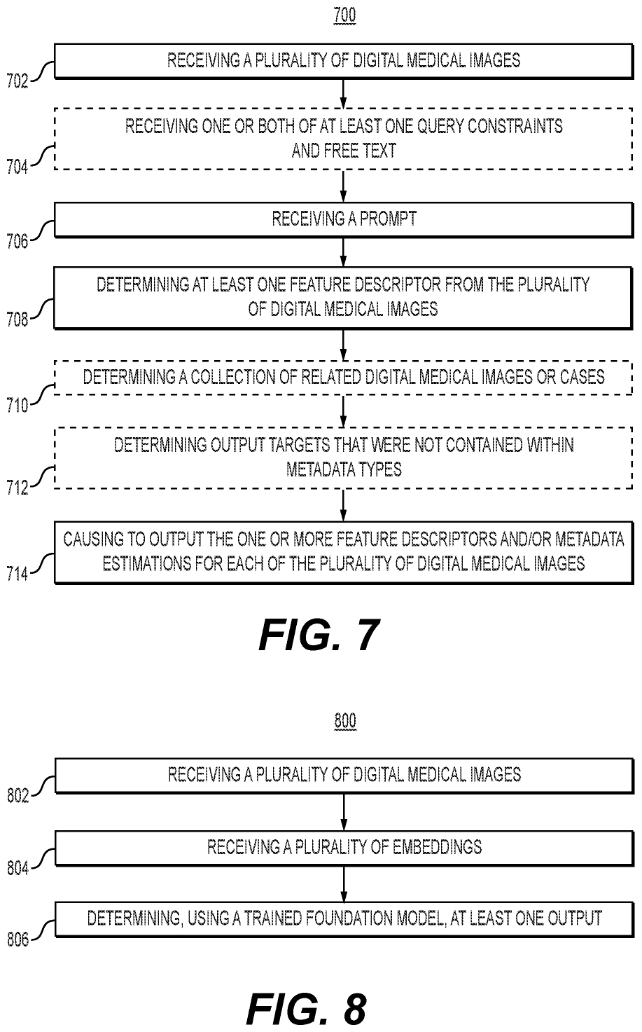

FIG. 7 depicts an exemplary method 700 for processing digital medical images to infer metadata from those images, according to one or more techniques. The metadata may include any combination of supplemental medical images, structured diagnostic reports, unstructured free text reports, genomic data, proteomic data, treatment data, responses, or diagnoses.

At step 702, a plurality of digital medical images may be obtained. For example, the plurality of digital medical images may be received from physician servers 121, hospital servers 122, clinical trial servers 123, research lab servers 124, laboratory information systems 125, etc.

Optionally, at step 704, one or both of at least one query constraints or free text may be received. In some techniques, the query constraints may include judgments or hypotheses from a clinician or expert.

At step 706, a prompt may be received. In some techniques, the prompt may be a request for a specific type of metadata to be inferred from the plurality of digital medical images.

At step 708, at least one feature descriptor may be determined from the plurality of digital medical images based on the prompt. In some techniques, the at least one feature descriptor may be determined using a foundation model. The foundation model may be trained using techniques described herein.

Optionally, at step 710, a collection of related digital images or cases may be determined. The collection of related digital images or cases may be based on the metadata associated with each of the digital medical images or cases. In some techniques, the collection of related digital images or cases may be determined using a content-based retrieval system. In some techniques, content-based constraints may be received. The content-based constraints may include instructions to include or exclude specific types of metadata, or attributes of that metadata, to a query for content retrieval.

Optionally, at step 712, at least one output target may be determined. The output targets may be determined using a downstream task model. The downstream task model may be trained using techniques described herein. In some techniques, the output targets may not be contained within metadata types, e.g., based on the one or more feature descriptors for each of the plurality of digital medical images. The output targets may include any combination of learning markers for drug response, building a model to replicate an existing biomarker, learning a novel biomarker from test data or other ground-truth indicators, or predicting additional disease states or diagnostics.

At step 714, the at least one feature descriptor, metadata estimations, and/or a structured synoptic diagnostic report for each of the plurality of digital medical images may be caused to be output. In some techniques, the metadata estimations may be consistent with the one or more query constraints. In some techniques, the structured synoptic diagnostic report may be based on the plurality of digital medical images and free text.

FIG. 8 depicts an exemplary method 800 for processing digital medical images to infer at least one embedding from those images, according to one or more techniques. At step 802, a plurality of digital medical images may be obtained. For example, the plurality of digital medical images may be received from physician servers 121, hospital servers 122, clinical trial servers 123, research lab servers 124, laboratory information systems 125, etc.

At step 804, a plurality of embeddings may be obtained. In some techniques, the plurality of embeddings may be obtained from a foundation model. The foundation model may be trained, e.g., for downstream tasks, using techniques described herein. The plurality of embeddings may be obtained for each of a plurality of modalities.

At step 806, at least one output may be determined. In some techniques, the at least one output may be determined using a trained foundation model (e.g., a trained downstream foundation model). The at least one output may include detecting a presence and/or score of biomarkers (e.g., HER2, KRAS, MSI, EGFR, etc.), detecting the presence, subtype, grade, origin site, etc. of the cancer in the WSI, predicting the overall survival, disease-free survival, and/or prognosis-free survival of the patient, predicting the response to a particular treatment, etc.

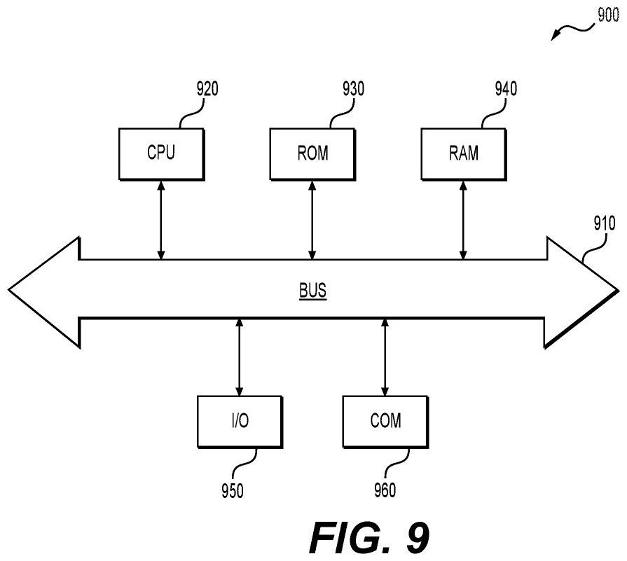

FIG. 9 illustrates an example computer-implemented system or device 900 that may execute techniques presented herein. Device 900 may include a central processing unit (CPU) 920. CPU 920 may be any type of processor device including, for example, any type of special purpose or a general-purpose microprocessor device. As will be appreciated by persons skilled in the relevant art, CPU 920 also may be a single processor in a multi-core/multiprocessor system, such system operating alone, or in a cluster of computing devices operating in a cluster or server farm. CPU 920 may be connected to a data communication infrastructure 910, for example a bus, message queue, network, or multi-core message-passing scheme.

Device 900 may also include a main memory 940, for example, random access memory (RAM), and also may include a secondary memory 930. Secondary memory 930, e.g. a read-only memory (ROM), may be, for example, a hard disk drive or a removable storage drive. Such a removable storage drive may comprise, for example, a floppy disk drive, a magnetic tape drive, an optical disk drive, a flash memory, or the like. The removable storage drive in this example reads from and/or writes to a removable storage unit in a well-known manner. The removable storage may comprise a floppy disk, magnetic tape, optical disk, etc., which is read by and written to by the removable storage drive. As will be appreciated by persons skilled in the relevant art, such a removable storage unit generally includes a computer usable storage medium having stored therein computer software and/or data.

In alternative implementations, secondary memory 930 may include similar means for allowing computer programs or other instructions to be loaded into device 900. Examples of such means may include a program cartridge and cartridge interface (such as that found in video game devices), a removable memory chip (such as an EPROM or PROM) and associated socket, and other removable storage units and interfaces, which allow software and data to be transferred from a removable storage unit to device 900.

Device 900 also may include a communications interface (COM) 960. Communications interface 960 allows software and data to be transferred between device 900 and external devices. Communications interface 960 may include a modem, a network interface (such as an Ethernet card), a communications port, a PCMCIA slot and card, or the like. Software and data transferred via communications interface 960 may be in the form of signals, which may be electronic, electromagnetic, optical or other signals capable of being received by communications interface 960. These signals may be provided to communications interface 960 via a communications path of device 900, which may be implemented using, for example, wire or cable, fiber optics, a phone line, a cellular phone link, an RF link or other communications channels.

The hardware elements, operating systems, and programming languages of such equipment are conventional in nature, and it is presumed that those skilled in the art are adequately familiar therewith. Device 900 may also include input and output ports 950 to connect with input and output devices such as keyboards, mice, touchscreens, monitors, displays, etc. Of course, the various server functions may be implemented in a distributed fashion on a number of similar platforms, to distribute the processing load. Alternatively, the servers may be implemented by appropriate programming of one computer hardware platform.

Throughout this disclosure, references to components or modules generally refer to items that logically may be grouped together to perform a function or group of related functions. Like reference numerals are generally intended to refer to the same or similar components. Components and/or modules may be implemented in software, hardware, or a combination of software and/or hardware.

The tools, modules, and/or functions described above may be performed by one or more processors. “Storage” type media may include any or all of the tangible memory of the computers, processors or the like, or associated modules thereof, such as various semiconductor memories, tape drives, disk drives and the like, which may provide non-transitory storage at any time for software programming.

Software may be communicated through the Internet, a cloud service provider, or other telecommunication networks. For example, communications may enable loading software from one computer or processor into another. As used herein, unless restricted to non-transitory, tangible “storage” media, terms such as computer or machine “readable medium” refer to any medium that participates in providing instructions to a processor for execution.

The foregoing general description is exemplary and explanatory only, and not restrictive of the disclosure. Other embodiments may be apparent to those skilled in the art from consideration of the specification and practice of the invention disclosed herein. It is intended that the specification and examples be considered as exemplary only.

What is claimed is:

1. A computer-implemented method for processing digital medical images to infer metadata from those images, the method comprising:
receiving a plurality of digital medical images;
receiving a prompt, the prompt being a request for a specific type of metadata to be inferred from the plurality of digital medical images;
determining, using a trained foundation model, at least one feature descriptor from the plurality of digital medical images based on the prompt; and
providing for output the at least one feature descriptor for each of the plurality of digital medical images.

2. The method of claim 1, wherein the plurality of digital medical images include at least one of whole slide image (WSI), hematoxylin and eosin (H&E) stains, immunohistochemistry (IHC) slides, immunofluorescent slides, or CT scans.

3. The computer-implemented method of claim 1, wherein the metadata comprises any combination of supplemental medical images, structured diagnostic reports, unstructured free text reports, genomic data, proteomic data, treatment data, responses, or diagnoses.

4. The computer-implemented method of claim 1, further comprising:
receiving at least one query constraint, the query constraints including judgments or hypotheses from a clinician or expert; and
providing for output, from the trained foundation model, metadata estimations that are consistent with the at least one query constraint.

5. The computer-implemented method of claim 1, further comprising:
receiving free text; and
providing for output, from the trained foundation model, a structured synoptic diagnostic report based on the plurality of digital medical images and free text.

6. The computer-implemented method of claim 1, further comprising:
determining, using a content-based retrieval system, a collection of related digital medical images or cases based on the metadata associated with each of the digital medical images or cases.

7. The computer-implemented method of claim 6, further comprising:
receiving content-based constraints, the content-based constraints comprising instructions to include or exclude specific types of metadata, or attributes of that metadata, to a query for content retrieval.

8. The computer-implemented method of claim 1, further comprising:
determining, using a downstream task model, output targets that were not contained within metadata types based on the at least one feature descriptor for each of the plurality of digital medical images.

9. The computer-implemented method of claim 8, wherein the output targets include any combination of learning markers for drug response, building a model to replicate an existing biomarker, learning a novel biomarker from test data or other ground-truth indicators, or predicting additional disease states or diagnostics.

10. A method of training a foundation model to process digital medical images to infer metadata from those images, the method comprising:
receiving a plurality of digital medical images;
generating a plurality of image tokens from the digital medical images, the image tokens being fixed-sized patches;
removing a subset of the plurality of image tokens from each of the digital medical images to generate a remaining plurality of image tokens from each of the digital medical images;
encoding, using an encoder, the remaining plurality of image tokens from each of the digital medical images;
adding a classification token to the encoded image tokens;
appending masked tokens with position encodings to each respective encoded image token; and
reconstructing, using a decoder, the image tokens, such that the image tokens align with original image pixel values.

11. The method of claim 10, wherein the encoder is a Vision Transformer (ViT) encoder.

12. The method of claim 10, wherein the classification token is a network-specific vector of numbers that summarizes an image tile representation.

13. The method of claim 10, wherein the decoder is a ViT decoder.

14. The method of claim 13, wherein the ViT decoder may be optimized using L2 image reconstruction loss applied on the masked tokens.

15. The method of claim 10, further comprising training the encoder and decoder to align the image tokens and the reconstructed masked tokens.

16. A system for processing digital medical images to infer metadata from those images, the system comprising:
at least one memory storing instructions; and
at least one processor configured to execute the instructions to perform operations comprising:
receiving a plurality of digital medical images;
receiving a prompt, the prompt being a request for a specific type of metadata to be inferred from the plurality of digital medical images;
determining, using a trained foundation model, at least one feature descriptor from the plurality of digital medical images based on the prompt; and
providing for output the at least one feature descriptor for each of the plurality of digital medical images.

17. The system of claim 16, wherein the plurality of digital medical images include at least one of whole slide images (WSI), hematoxylin and eosin (H&E) stains, immunohistochemistry (IHC) slides, immunofluorescent slides, or CT scans.

18. The system of claim 16, wherein the metadata comprises any combination of supplemental medical images, structured diagnostic reports, unstructured free text reports, genomic data, proteomic data, treatment data, responses, or diagnoses.

19. The system of claim 16, the operations further comprising:
determining, using a downstream task model, output targets that were not contained within metadata types based on the at least one feature descriptor for each of the plurality of digital medical images.

20. The system of claim 19, wherein the output targets include any combination of learning markers for drug response, building a model to replicate an existing biomarker, learning a novel biomarker from test data or other ground-truth indicators, or predicting additional disease states or diagnostics.
